(19) United States
(12) Patent Application Publication
(10) Pub. No.: US 2011/0270048 A1
(43) Pub. Date: Nov. 3, 2011

(54) SYSTEMS AND METHODS FOR PPG SENSORS INCORPORATING EKG SENSORS

(75) Inventors: Paul Stanley Addison, Edinburgh (GB); James N. Watson, Dunfermline (GB); Rakesh Sethi, Vancouver (CA)

(73) Assignee: Nellcor Puritan Bennett Ireland, Mervue (IE)

(21) Appl. No.: 12/771,770

(22) Filed: Apr. 30, 2010

(51) Int. Cl. A61B 5/02; A61B 5/021

(52) U.S. Cl. 600/301; 600/485

(57) ABSTRACT

Techniques and structures are disclosed for using photoplethysmograph (PPG) and electrocardiographic (EKG)-based readings of a subject to determine one or more physiological characteristics of the subject. In an arrangement, a combined PPG-EKG sensor unit may be used to detect both PPG and EKG signals of the subject. The sensor unit may include a PPG sensor, an EKG sensor, and a support structure for connecting or fastening the sensor unit to the subject. The detected readings may be provided to an electronic monitor. In an arrangement, a PPG-EKG monitoring system, including the electronic monitor, may be used to determine the physiological parameters of the subject. The monitoring system may first determine an auxiliary parameter based at least in part on the EKG signal, and then compute the one or more physiological characteristics of the subject based at least in part on both the PPG signal and the auxiliary parameter.

### SUMMARY

The present disclosure is related to signal processing systems and methods, and more particularly, to systems and methods for detecting one or more physiological characteristics of a subject using one or more combined photoplethysmograph (PPG)-electrocardiographic (EKG) sensor units.

In an arrangement, a PPG-EKG sensor unit is used to detect signals related to physiological characteristics of a subject. The sensor unit includes a PPG sensor for detecting a PPG signal, an EKG sensor detecting an EKG signal, and an interface for relaying the detected PPG and EKG signals to one or more electronic monitors. In an arrangement, the PPG sensor includes pulse oximetry components, such as an LED emitter and a photoelectric detector. In an arrangement, the EKG sensor may correspond to a passive detecting element such as a metal electrode.

In an arrangement, the sensor unit is connected (or otherwise affixed or fastened) to a digit, appendage (e.g., an ear), or other body part of the subject, and includes a support structure to hold the sensor unit to the top and bottom sides of the subject's digit or appendage (e.g., an ear), or to the surface of a body part. The support structure may include multiple components that are connected directly or indirectly. The support structure need not be a single continuous substance or piece, and may be used to keep PPG sensors and EKG sensors structurally connected. For example, the support structure may physically bind to PPG and EKG sensors. The support structure may include one or more cables connected to the PPG and EKG sensors and may receive and/or hold these cables in place. The sensor unit may include a spring and padding material, and the padding material may be adjustable to conform to, for example, the size and shape of the subject's digit, appendage (e.g., an ear), or other part. In an arrangement, the sensor unit includes an adhesive layer that may be affixed onto a portion of the body of the subject. In an arrangement, the sensor unit includes an interface, and the interface may be a cable interface or a wireless interface. In an arrangement, a common cable (possibly including two or more separate wires) is used by the sensor unit to provide both the detected PPG signal and the detected EKG signal to the electronic monitors. In an arrangement, the sensor unit uses separate cables to provide the detected PPG and EKG signals to one or more electronic monitors.

A monitoring system may be used to determine one or more physiological parameters of the subject based at least in part on the detected PPG and EKG signals. In an arrangement, the monitoring system determines an auxiliary parameter based at least in part on the detected EKG signal, and determines the one or more physiological parameters based at least in part on the detected PPG signal in combination with the auxiliary parameter. In an arrangement, the auxiliary parameter may be a trigger or trigger indicator, and may be used to trigger an ensemble averaging of the detected PPG signal. The auxiliary parameter may be an indicator of the presence of an arrhythmia condition in the subject, and/or may relate to respiration information determined based at least in part on impedance changes of the subject.

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 is a perspective view of an arrangement of a subject monitoring system using multiple combined PPG-EKG sensors;

FIG. 2 is a detailed arrangement of a subject monitoring system using multiple combined PPG-EKG sensors;

FIG. 3 is another detailed arrangement of a subject monitoring system using multiple combined PPG-EKG sensors;

FIGS. 4A-4C depict illustrative arrangements of combined PPG-EKG sensors that may be used in a monitoring system such as monitoring system of FIGS. 1-3; and

FIG. 5 depicts an illustrative cross-sectional view of a combined PPG-EKG sensor that may be used in a monitoring system such as the monitoring systems of FIGS. 1-3.

### DETAILED DESCRIPTION

Monitoring the physiological state of a subject, for example, by determining, estimating, and/or tracking one or more physiological parameters of the subject, may be of interest in a wide variety of medical and non-medical applications. Knowledge of a subject's physiological characteristics (e.g., through a determination of one or more physiological parameters such as blood pressure, oxygen saturation, and presence of specific heart conditions) can provide short- and long-term benefits to the subject, such as early detection and/or warning of potentially harmful conditions, diagnosis and treatment of illnesses, and/or guidance for preventative medicine.

Physiological parameters of a subject can be determined from a photoplethysmograph (PPG) signal, and such a signal can be obtained from a subject using a PPG sensor and a wide variety of suitable techniques. For example, a PPG signal can be obtained from a subject using a PPG sensor in the form of a pressure transducer that may be fastened to subject's wrist area. Alternatively, a PPG signal can be obtained using a PPG sensor in the form of a pulse oximetry sensor that is clipped to a digit, appendage (e.g., an ear), or other part of the subject (the term “digit” refers herein to a toe or finger of a subject). Such a PPG sensor may be used to determine the blood oxygen saturation of a subject.

Further, a second PPG sensor may be affixed to a subject, and the combination of these two PPG sensors may allow for the determination of the subject's blood pressure, for example, using continuous non-invasive blood pressure (CNIBP) techniques. For example, in an arrangement, two PPG-based pulse oximetry sensors can be used. One of these sensors may be used to determine the blood oxygen saturation of the subject, and/or both sensors may be used in combination to determine an estimate of the blood pressure of the subject via non-invasive techniques.

The use of two PPG sensors, for example, two pulse oximeter sensors, for the measurement of oxygen saturation, blood pressure, and/or other physiological parameters may also allow for the measurement of an EKG signal or signals. For example, in an arrangement, an EKG sensor (e.g., an electrode) may be placed in or near each PPG sensor. For example, in an arrangement, each PPG sensor may be a pulse oximetry sensor, and an EKG sensor may be placed within the housing of each of these PPG sensors. In general, a sensor configuration including both PPG sensors and EKG sensors may be advantageously used to detect a PPG signal or signals in combination with an EKG signal or signals, and may provide a range of useful information regarding a subject. For example, in an arrangement, one or more physiological parameters of a subject may be determined using PPG sensors (such as pulse oximetry sensors, CNIBP sensors, and/or pressure transducer sensors) combined with EKG sensors to produce weighted biosignal information. In an arrangement, measurements made by each of these PPG sensors may be combined with measurements made by EKG sensors (e.g., EKG electrodes) to, for example, be used as a gating signal for determining a subject oxygen saturation level. In an arrangement, a peak of a R-wave in a measured EKG signal or signals may be used for the filtering of one or more oxygen saturation signals. In an arrangement, a filtering process may be used to, for example, trigger an ensemble averaging of at least two of the measured PPG signals, which may improve the derivation of physiological and/or biosignal parameters.

In an arrangement, a PPG sensor may be affixed to a subject. As described above, this PPG sensor may correspond to a pulse oximetry sensor (and may be used as a single sensor to determine a blood oxygen saturation level, and/or as one of two sensors in tandem to determine a subject blood pressure). The PPG sensor may emit light that is passed through the tissue of a subject and detected by a detector. The light passed through the tissue may be selected to be of one or more wavelengths that are absorbed by the subject's blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambert-Beer's law. The following notation will be used herein:

I(λ,t)=I_{O}(λ)exp(−(sβ_{O}(λ)+(1−s)βᵣ(λ))l(t)) (1)

where:
λ=wavelength;
t=time;
I=intensity of light detected;
Iₒ=intensity of light transmitted;
s=oxygen saturation;
βₒ, βᵣ=empirically derived absorption coefficients; and
l(t)=a combination of concentration and path length from emitter to detector as a function of time.

PPG monitor 250 may measure light absorption at two wavelengths (e.g., red and infrared (IR)), and then calculate saturation by solving for the “ratio of ratios” as follows.

1. First, the natural logarithm of (1) is taken (“log” will be used to represent the natural logarithm) for IR and Red

log I=log Iₒ−(sβₒ+(1−s)βᵣ)l (2)

2. (2) is then differentiated with respect to time

$$\frac{d log I}{d t} = - ( s {β}_{o} + ( 1 - s ) {β}_{r} ) \frac{d l}{d t} \qquad ( 3 )$$

3. Red (3) is divided by IR (3)

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} \qquad ( 4 )$$

### 4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\frac{d log I ( {λ}_{R} )}{d t} ( {β}_{o} ( {λ}_{IR} ) - {β}_{r} ( {λ}_{IR} ) ) - \frac{d log I ( {λ}_{IR} )}{d t} ( {β}_{o} ( {λ}_{R} ) - {β}_{r} ( {λ}_{R} ) )}$$

Note in discrete time

$$\frac{d log I ( λ , t )}{d t} ≃ log I ( λ , {t}_{2} ) - log I ( λ , {t}_{1} )$$

### Using log A−log B=log A/B,

$$\frac{d log I ( λ , t )}{d t} ≃ log ( \frac{I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} - {λ}_{R} )}{I ( {t}_{2} , {λ}_{R} )} )}{log ( \frac{I ( {t}_{1} , {λ}_{IR}}{I ( {t}_{2} , {λ}_{IR}} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} .$$

From (5), R can be calculated using two points (e.g., PPG maximum and minimum), or a family of points. One method using a family of points uses a modified version of (5). Using the relationship

$$\frac{d log I}{d t} = \frac{\frac{d I}{d t}}{I} \qquad ( 6 )$$

now (5) becomes

$$\frac{\frac{d log I ( {λ}_{R} )}{d t }}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR } ) - I ( {t}_{1} , {λ}_{IR} )}{I ( {t}_{1} , {λ}_{IR} )}} = \frac{[ I ( {t}_{2} , {λ}_{R} ) - I ( {t}_{1} , {λ}_{R } ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} = R \qquad ( 7 )$$

which defines a cluster of points whose slope of y versus x will give R where

$$x ( t ) = [ I ( {t}_{2} , {λ}_{IR} ) - I ( {t}_{1} , {λ}_{IR} ) ] I ( {t}_{1} , {λ}_{R} ) \\ y ( t ) = [ I ( {t}_{2} , {λ}_{R} ) - I ( {t}_{1} , {λ}_{R} ) ] I ( {t}_{1} , {λ}_{IR} ) \\ y ( t ) = Rx ( t ) \qquad ( 8 )$$

Once R is determined or estimated, for example, using the techniques described above, the blood oxygen saturation can be determined or estimated using any suitable technique for relating a blood oxygen saturation value to R. For example, blood oxygen saturation can be determined from empirical data that may be indexed by values of R, and/or it may be determined from curve fitting and/or other interpolative techniques.

In an arrangement, two PPG sensors may be affixed to a subject. As described above, these two PPG sensors may correspond to two pulse oximetry sensors, and may be used to determine a CNIBP of a subject. Each sensor may be positioned at different locations on a subject's body to estimate the blood pressure and/or other related biosignal parameters of the subject from a measured signal or signals. In an arrangement, a reference point of a measured signal may be identified (and this reference point may correspond to a reference “feature,” such as a leading or trailing edge of the signal, or the location of a signal peak or valley), and the elapsed time, denoted T, between the arrival times of this reference point at the two sensors (e.g., pulse oximetry sensors) may be determined. An estimate of the subject's blood pressure, p, may then be determined from any suitable relationship between the blood pressure and T. For example, in an arrangement, the following mathematical relation may be used to determine an estimate of subject blood pressure from the elapsed time

p=a+b·ln(T),

where a and b are constants that may be determined from a calibration process and may be dependent on the nature of the subject and signal detector that are, for example, affixed to the subject. Once calibration has been completed, for example, using a non-invasive blood pressure device, an equation similar or identical to the one above can be used to determine a subject blood pressure. The equation above is meant to be illustrative, and any other suitable equation (or equations) may also be used to derive an estimated subject blood pressure. Further, blood pressure estimates may be computed on a continuous or periodic basis. For example, Chen et al. U.S. Pat. No. 6,599,251, which is hereby incorporated by reference herein in its entirety, discloses some techniques for continuous and non-invasive blood pressure monitoring using two probes or sensors that may be used in conjunction with the present disclosure.

The use of PPG signals combined with EKG signals may be advantageous in detecting and/or analyzing arrhythmias of a subject (e.g., a subject in a medical setting). In an arrangement, the EKG sensor may be used to detect an auxiliary parameter, such as, for example, an indication of an arrhythmia of a subject. The detection of this auxiliary parameter (e.g., the indication of the arrhythmia) may then be used to partially or fully determine a signal quality and/or confidence level in a (parallel) calculation performed based at least in part on PPG sensor readings. Arrhythmia detection using a monitoring system that includes combined PPG-EKG sensors may be advantageous in providing an early warning and/or detection of an arrhythmia onset that is more accurate than would be possible using EKG sensors alone. Similarly, the detection of biosignal and/or physiological parameters, for example, related to the blood pressure of a subject, may be done more accurately using a combined PPG-EKG sensor system than would be possible if only PPG sensors were used. Further, such a combined PPG-EKG monitoring system may be used in settings where a standard PPG- or EKG-sensor setup would be inconvenient and/or cost-prohibitive. Some of the EKG-sensor setups described herein may result in the EKG sensors being placed in atypical locations for EKG leads. However, the EKG-sensor setups described herein may still provide a range of information that is useful for determining a characteristic or characteristics of a subject.

In an arrangement, a combined PPG-EKG sensor system may be advantageously used to derive a subject respiration parameter, for example, a subject respiration rate, through EKG-based detected impedance changes (in this case, the detected impedance changes may correspond to the EKG-derived auxiliary parameter). Impedance changes may be detected using techniques similar or identical to those used to measure impedance in respiration rate monitoring. For example, impedance changes may be detected using one or multiple Trans-Thoracic Impedance (ITT) measurements. In an arrangement, impedance changes may be measured by one or multiple EKG electrodes which measure changes in the impedance across the chest wall, which correspond to cyclical changes in tidal volume (e.g., due to a patient respiration). Such an EKG respiratory signal may be used to determine respiratory effort. In an arrangement, an EKG respiratory signal, obtained from the EKG signals, may be combined with a PPG signal, derived from PPG sensors, to determine these and/or other suitable parameters.

PPG and EKG signals derived from a combined PPG-EKG sensor system may be advantageously combined using any suitable technique. For example, in an arrangement, a respiration rate measurement, derived from one or multiple EKG sensors, may be combined with a second independent respiration rate measurement, derived from one or multiple PPG sensors. Further, in an arrangement, these two signals may be combined using a weighted average, where the weights are dependent, at least partially, on an actual or measured reliability or integrity reading associated with the signal. In an arrangement, information associated with one or multiple measurements of a respiration effort, an indication of change in a respiratory effort, or any suitable combination thereof, may be combined in a useful way (e.g. by weighing multiple measurements of information according at least to one or more signal quality metrics). In an arrangement, the EKG signal may be used to identify the current heart rate and quantify respiratory sinus arrhythmia (RSA) which may be used in the determination of respiratory effort together with the PPG pulse amplitude.

In an arrangement, the EKG signal may be use to detect subject movement. In an arrangement, movement may be detected from one or multiple EKG-based signals by detecting signal baseline shifts. In an arrangement, signal baseline shifts may be used to identify corrupt segments of a PPG signal (e.g., due to signal noise). Further, noisy or corrupt segments may be reduced using noise mitigation strategies such as, for example, changing filter characteristics, using respiratory information from signal components unaffected by noise, or holding a last good signal value until the current signal is no longer corrupt or noisy. In an arrangement, movement detections may be used in the identification and/or filtering of noise from the PPG signal detected at one or more sensors. In an arrangement, one or multiple EKG signal measurements may be used to trigger an ensemble average of one or more PPG signal measurements to reduce or eliminate signal corruption or noise. Combined PPG-EKG sensors identical or similar to those described may be useful in detecting or analyzing a wide range of physiological parameters including oxygen saturation, heart rate, EKG, respiration rate, respiration effort, blood pressure, arrhythmias, other suitable parameters, and/or any suitable combination thereof.

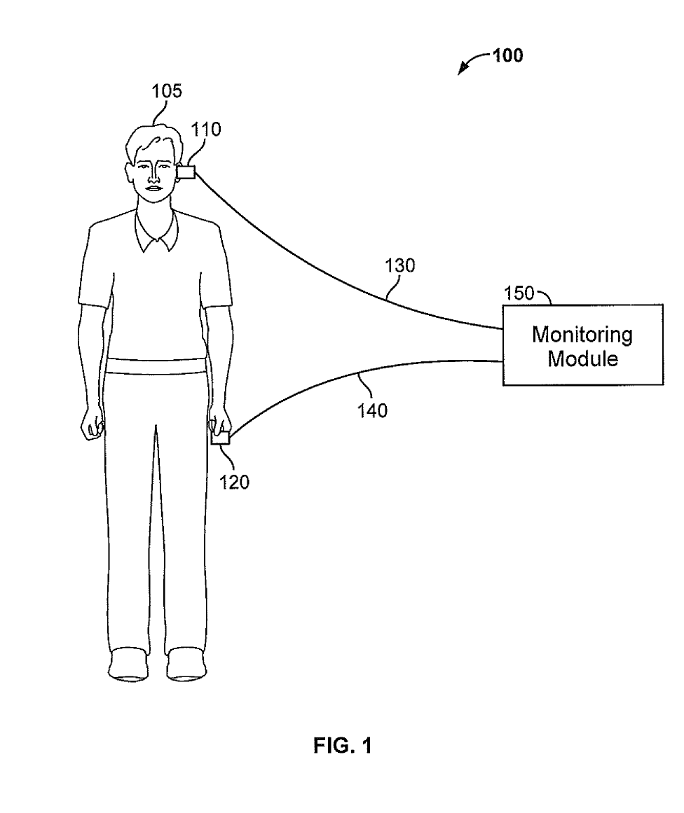

FIG. 1 is a perspective view of an arrangement of a subject monitoring system using multiple PPG-EKG sensors (i.e., multiple sensors, each including a PPG detecting mechanism, such as a PPG sensor, and an EKG detecting mechanism, such as an EKG sensor). Monitoring system 100 may use two sensors, for example, PPG-EKG sensor 110 and PPG-EKG sensor 120, to measure biological characteristics of a subject such as subject 105. Subject 105 may correspond a human subject and may correspond to a human of any age, sex, or other suitable demographic. Additionally, monitoring system 100 may be used in a medical setting, and further, subject 105 may correspond to a medical patient. Alternatively, monitoring system 100 be used in a home or other informal setting. In an embodiment, monitoring system 100 may be operated directly by subject 105. Alternatively, monitoring system 100 may be operated by a human or non-human operator, including, for example, by a medical professional not pictured in FIG. 1. In an arrangement, sensors 110 and 120 may be connected to a monitoring module such as monitoring module 150 using cables such as cable 130 and/or cable 140. Alternatively, one or both of sensors 110 and 120 may be connected to monitoring module 150 via a wireless link. In an arrangement, sensor 110 and/or 120 may draw power from monitoring module 150. In an arrangement, sensor 110, sensor 120, cable 130, and/or cable 140 may include circuitry and/or processors to pre-process or filter a measured signal. In an arrangement, such circuitry may perform noise-cancellation and/or any other suitable filtering operation. In an arrangement, cables 130 and 140 may be shielded to reduce or eliminate cross-talk and/or various other forms of electrical interference. Each of the cables disclosed herein, including, for example, each of cables 130 and 140, may contain or otherwise enclose any suitable number of wires. For example, each of cables 130 and 140 may include two or more wires in the form of a twisted wire pair or bundled twisted wire pairs. In an arrangement, cable 130 (and similarly, cable 140) may include two wires, with one wire serving as a “ground” wire. Further, cable 130 (and similarly, cable 140) may include more than two wires, where one or more of these wires is a twisted pair. In this configuration, one of the wires may carry information based at least in part on the signal detected by a PPG sensor, and the other wire may carry information based at least in part on the signal detected by the EKG sensor.

In an arrangement, sensor 110 and/or 120 may be placed at positions of a subject other than those shown in FIG. 1. For example, in an arrangement, one or both of these sensors may be positioned to detect pulsatile flow of a subject. To this end, one or both of sensors 110 and 120 may be placed, for example, over an artery or on any other suitable location of a subject. Sensor 110 and/or 120 may be placed on a digit, that is, on a finger or toe of a subject, or may be placed on any other suitable appendage or body part of the subject. As described herein, the “top of a digit” will refer to the top side of a digit, that is, the side including the full width of the subject's (finger or toe) nail when the subject's digit is laid flat. Further, “bottom of a digit” will refer to the side that does not include the subject's (finger or toe) nail when the subject's digit is laid flat. In an arrangement, the placement of one or both of sensors 110 and 120 may be subject specific and/or may be determined through a trial-and-error process and/or through the use of calibration measurements.

In an arrangement, one or both of sensors 110 and 120 may be placed to optimize the detection and/or determination of CNIBP and/or arrhythmias of a subject. In an arrangement, more than two sensors may be used by monitoring system 100. For example, additional probes may be used that are similar or identical to traditional EKG probes. In an arrangement, a total of 10 or 12 probes may be used to detect one or more EKG signals, and only a certain number of these probes, for example, two probes, may contain PPG-capable sensors (while some or all of the probes may contain EKG-capable sensors). It will be understood that each sensor used in monitoring system 100 (e.g., sensor 110 and/or sensor 120) may represent a single integrated PPG-EKG sensor, a PPG sensor and an EKG sensor combined into a single housing, separate PPG and EKG sensors provided in separate housing, and/or any other suitable configuration of PPG and EKG-detecting devices.

In an arrangement, monitoring module 150 receives and combines signals received from sensor 110 and/or 120 to produce data useful for determining and/or interpreting a physiological state of subject 105. For example, in an arrangement, monitoring module 150 may receive PPG and EKG signals from sensor 110, which may be located, for example, on or near the ear of a subject (sensor 110 may generally be located on any suitable appendage, digit, or body part of the subject), and different PPG and EKG signals from sensor 120, which may be located, for example, on a digit of the subject. Monitoring module 150 may combine these signals using any suitable technique. For example, monitoring module 150 may linearly weight the received signals to produce a single overall signal from which a physiological characteristic is determined, or it may perform calculations directly on the received PPG signals and/or EKG signals.

Monitoring system 100 may be used to determine one or more physiological parameters of the subject based at least in part on the detected PPG and EKG signals. In an arrangement, monitoring system 100 may determine an auxiliary parameter based at least in part on the detected EKG signal, and may then determine one or more physiological parameters based at least in part on the detected PPG signal in combination with the auxiliary parameter. In an arrangement, the auxiliary parameter may be a trigger or trigger indicator, and may be used to trigger an ensemble averaging of the detected PPG signal. Additionally or alternatively, the auxiliary parameter may be an indicator of the presence of an arrhythmia condition in the subject, and/or may relate to respiration information determined based at least in part on impedance changes of the subject. The auxiliary parameter may generally correspond to a weight or weighting, a probability or confidence level, and/or to any other suitable type of data fusion metric that may be useful in evaluating the quality, precision, and/or accuracy of the detected PPG signal (and/or of any statistic derived from the detected PPG signal).

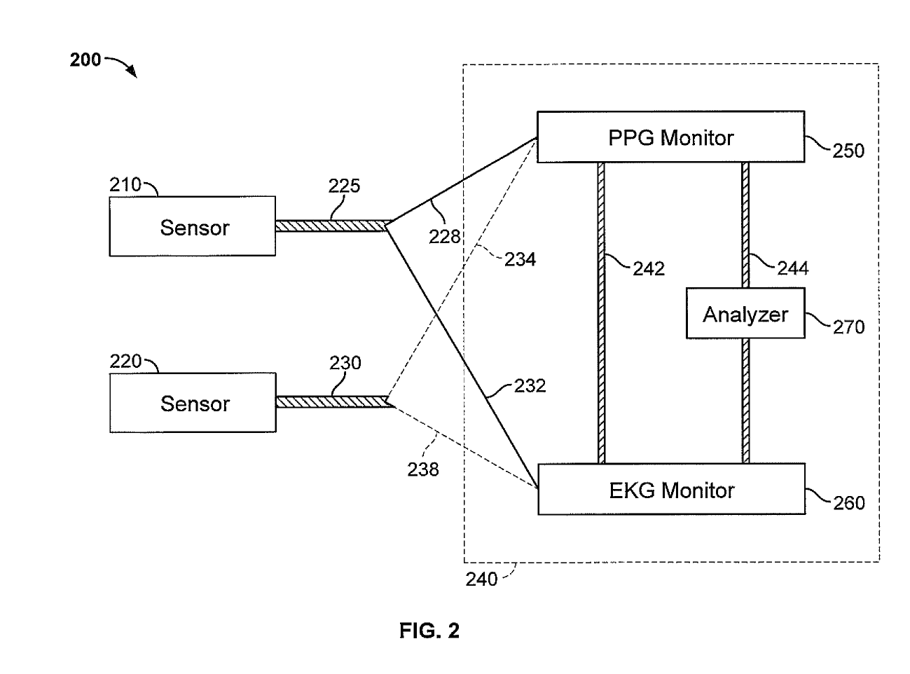

FIG. 2 is a detailed arrangement of a subject monitoring system using multiple combined PPG-EKG sensors. Monitoring system 200 may correspond to a more detailed description of monitoring system 100 (FIG. 1) in accordance with an arrangement. For example, sensor 210, sensor 220, cable 225, cable 230, and monitoring module 240 may correspond to sensor 110 (FIG. 1), sensor 120 (FIG. 1), cable 130 (FIG. 1), cable 140 (FIG. 1), and monitoring module 150 (FIG. 1), respectively, in accordance with an arrangement. As depicted in FIG. 2, cable 225 (and similarly, cable 230) may include two separate subcables, for example, subcables 228 and 232. In an arrangement, subcable 228 may transmit a PPG signal to PPG monitor 250, while subcable 232 may transmit an EKG-related signal to EKG monitor 260. In an arrangement, cable 225 (and similarly, cable 230) may be shielded or screened to reduce or prevent interference between constituent subcables 228 and 232. For example, the shielding included in cable 225 (and similarly, the shielding including in cable 230) may act as a Faraday cage to reduce electrical interference and/or cross-talk within and external to the cable.

As shown in FIG. 2, monitoring module 240 may include PPG monitor 250 and EKG monitor 260. In an arrangement, PPG monitor 250 may operate similarly or identically to PPG monitors as described in Watson et al., U.S. application Ser. No. 12/437,326 filed May 7, 2009, entitled “Consistent Signal Selection by Signal Segment Selection Techniques,” (Attorney Docket Reference: COV-42) which is incorporated by reference herein in its entirety. PPG monitor 250 may, for example, indirectly measure the oxygen saturation of a subject's blood and changes in blood volume in the skin. PPG monitor 250 may also measure the pulse rate of the subject. In an arrangement, PPG monitor 250 may measure and display various blood flow characteristics including, but not limited to, the oxygen saturation of hemoglobin in arterial blood. Alternatively or additionally, PPG monitor 250 may calculate a subject's blood pressure and/or other related parameters. For example, PPG monitor 250 may compute blood pressure using CNIBP-based techniques. PPG monitor 250 may display the results of any of these calculation on one or more suitable display screens and/or monitors.

Each of sensor 210 and sensor 220 may include, as part of the PPG-detecting capability, a light emitter and detector. For example, in an arrangement, sensor 210 may emit light through blood perfused tissue using the light emitter and photoelectrically sense the absorption of light in the tissue using the light detector. Sensor 210 may also measure the intensity of light that is received at the light detector as a function of time (sensor 220 may operate similarly or identically to sensor 210, while placed at a different location on a subject (such as subject 105 (FIG. 1))). A signal representing the measured light intensity versus time or a mathematical manipulation of this signal (e.g., a scaled version thereof, a log taken thereof, a scaled version of a log taken thereof, etc.) may be referred to as the measured PPG signal. The term PPG signal may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

As shown in FIG. 2, monitoring module 240 may include EKG monitor 260. EKG monitor 260 may be used with one or more EKG sensors to measure the electrical activity of a subject's heart. For example, by placing EKG sensors (e.g., electrodes) at locations on subject 105 (FIG. 1), measurements of electrical activity may be obtained. In an arrangement, sensors 210 and 220 may each include an EKG sensor that is affixed onto a subject using adhesive pads with sufficiently high electrical conductivity, and subcables 232 and 238 may be used to carry electrical signals from sensors 210 and 220, respectively, to EKG monitor 260.

EKG monitor 260 may process received signals, for example, the signals received through subcables 232 and 238 to detect potential arrhythmias and/or other heart related conditions. EKG monitor 260 may include one or more filters that may be used to process the received signals. For example, EKG monitor 260 may include a low-frequency filter (or filters) and/or a high-frequency filter (or filters) to isolate the received signals from noise components and/or to incorporate a priori knowledge of the signal parameters that are to be detected.

In an arrangement, the data of PPG monitor 250 and EKG monitor 260 may be combined or “fused” using an analyzer such as analyzer 270. In an arrangement, analyzer 270 may combine or weigh processed or unprocessed data from PPG monitor 250 and EKG monitor 260 to, for example, determine one or more biosignal parameters. For example, if a subject arrhythmia is detected by EKG monitor 260, this information may be used to adjust a quality setting or confidence metric affecting the determination of a signal parameter (e.g., a subject respiration rate) based at least in part on a PPG signal received and processed by PPG monitor 250.

It will be understood that the described components of monitoring module 240, including PPG monitor 250, EKG monitor 260, and analyzer 270, may be separate components connected by cables such as cable 242 and/or cable 244 (and that these cables may include insulating shielding to limit electrical interference and/or cross-talk present in monitoring system 200). Alternatively or additionally, one or more of these components may be housed in a common enclosure such as a metal or plastic enclosure. In an arrangement, one or more of these components may be manufactured in an integrated system. For example, PPG monitor 250, EKG monitor 260, and/or analyzer 270 may be implemented on a single piece of computer hardware and/or may share common system resources, including one or more common processors and/or memory storage units.

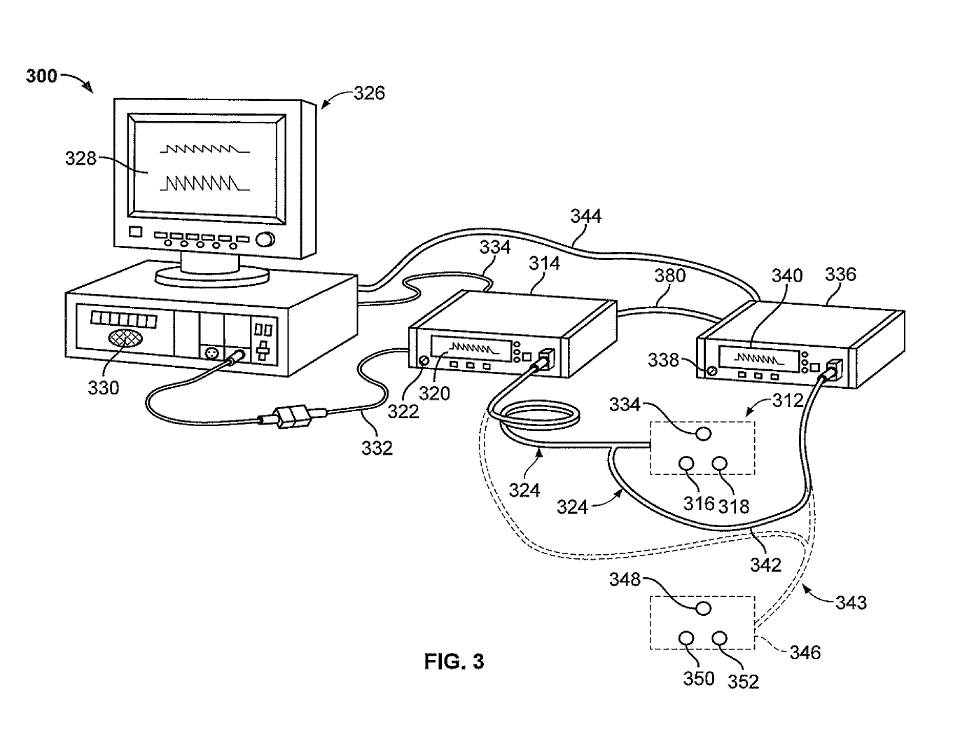

FIG. 3 is another detailed arrangement of a subject monitoring system using multiple combined PPG-EKG sensors. Monitoring system 300 may correspond to, for example, a further specification of monitoring system 100 (FIG. 1). Monitoring system 300 may include PPG monitor 314 and EKG monitor 336, which may correspond to PPG monitor 250 and EKG monitor 260 (both of FIG. 2), respectively. Monitoring system 300 may include cables 324 and 343, which may correspond to cables 225 and 230 (both of FIG. 2), respectively. Monitoring system 300 may include sensors 312 and 346, which may correspond to sensors 210 and 220 (both of FIG. 2), respectively. In an arrangement, each of sensors 312 and 346 may be a PPG-EKG sensor capable of detecting a PPG signal and an EKG signal. For example, sensor 312 may include emitter 316 for emitting light at a single wavelength (possibly with some dispersion into a continuum of wavelengths) into a subject's (e.g., subject 105 (FIG. 1)) tissue, and detector 318 for detecting the light that emanates from the subject's tissue. Alternatively, emitter 316 may emit light at two, or more, wavelengths (again, possibly with some dispersion into a continuum of wavelengths) into a subject's tissue. For example, emitter 316 may emit light at two wavelengths if emitter 316 is an emitter designed for pulse oximetry applications. Sensor 346 may include emitter 350 and detector 352, which may operate similarly or identically to emitter 316 and detector 318, respectively. Each of sensors 312 and 346 may include an EKG sensor to detect electrical signals of a subject. For example, sensors 312 and 346 may include EKG sensors 334 and 348, respectively, to detect an EKG signal or signals of a subject such as subject 105 (FIG. 1). In an arrangement, EKG sensors 334 and 348 may be affixed onto a subject using adhesive pads with sufficiently high electrical conductivity to permit an accurate EKG signal reading. Such adhesive pads may be designed for long-term use. Alternatively, such adhesive pads may be designed to be disposable and replaced, for example, after each use.

A part of each sensor used in monitoring system 300, for example, a part of sensor 312 and sensor 346, may be made of complementary metal oxide semiconductor (CMOS) material. Alternatively or additionally, a part of sensor may be made out of coupled device (CCD) material. In an arrangement, each sensor may include both CMOS and CCD constituent components. The CCD component of a sensor included in monitoring system 300 may comprise a photoactive region and a transmission region for receiving and transmitting data whereas the CMOS sensor may be made up of an integrated circuit having an array of pixel sensors. Each pixel may have a photodetector and an active amplifier. In an arrangement of a sensor, an emitter (e.g., emitter 316 of sensor 312) and detector (e.g., detector 318 of sensor 312) may be on opposite sides of a subject's digit such as a finger or toe, in which case the light that is emanating from the tissue has passed completely through the digit. In an approach, emitter 316 and detector 318 may be arranged so that light from emitter 316 penetrates the tissue and is reflected by the tissue into detector 318. An example of such a sensor may be a sensor designed to obtain pulse oximetry data from a subject's forehead.

Multi-parameter subject monitor 326 may be configured to receive signals from PPG monitor 314 and EKG monitor 336, calculate physiological parameters based at least in part on these signals, and provide a display of these signals on, for example, display 328. In an arrangement, multiparameter subject monitor 326 may be configured to display an estimate of a subject's blood pressure and blood oxygen saturation (referred to as “SpO₂”) generated by PPG monitor 314, and a confidence measure of one or both of these estimates derived from EKG monitor 336. In an arrangement, multi-parameter subject monitor 326 may include a speaker such as speaker 330 to sound an audible alarm, for example, in the event that a subject's physiological parameters are not within a predefined normal range.

PPG monitor 314 and/or EKG monitor 336 may be communicatively coupled to multi-parameter subject monitor 326 using a combination of cables such as cables 332, 334, and/or 344. Alternatively or additionally, PPG monitor 314 and/or EKG monitor 336 may be coupled to a sensor input port or a digital communications port, respectively and/or may communicate wirelessly (not shown) to multi-parameter subject monitor 326. In addition, PPG monitor 314 and/or EKG monitor 336 may be coupled to a network to enable the sharing of information with servers or other workstations (not shown). In an arrangement, PPG monitor and EKG monitor 336 may be directly communicatively coupled using a cable such as cable 380. PPG monitor 314 and/or EKG monitor 336 may be powered by a battery (not shown) or by a conventional power source such as a wall outlet. In an arrangement, PPG monitor 314 and/or EKG monitor 336 may be configured to calculate physiological parameters based at least in part on data received from sensors 312 and/or 346.

In an arrangement, PPG monitor 314 and/or EKG monitor 336 may include displays 320 and 340, respectively, to display subject physiological parameters and/or other information about a monitoring system. Each of display 320 and display 340 may be cathode ray tube type, a flat panel display (as shown) such as a liquid crystal display (LCD) or a plasma display, or any other suitable type of monitor. Further, PPG monitor 314 and/or EKG monitor 336 may include speakers 322 and 338, respectively, to provide an audible sound that may be used in various arrangements, such as for example, sounding an audible alarm in the event that a subject's physiological parameters are not within a predefined normal range.

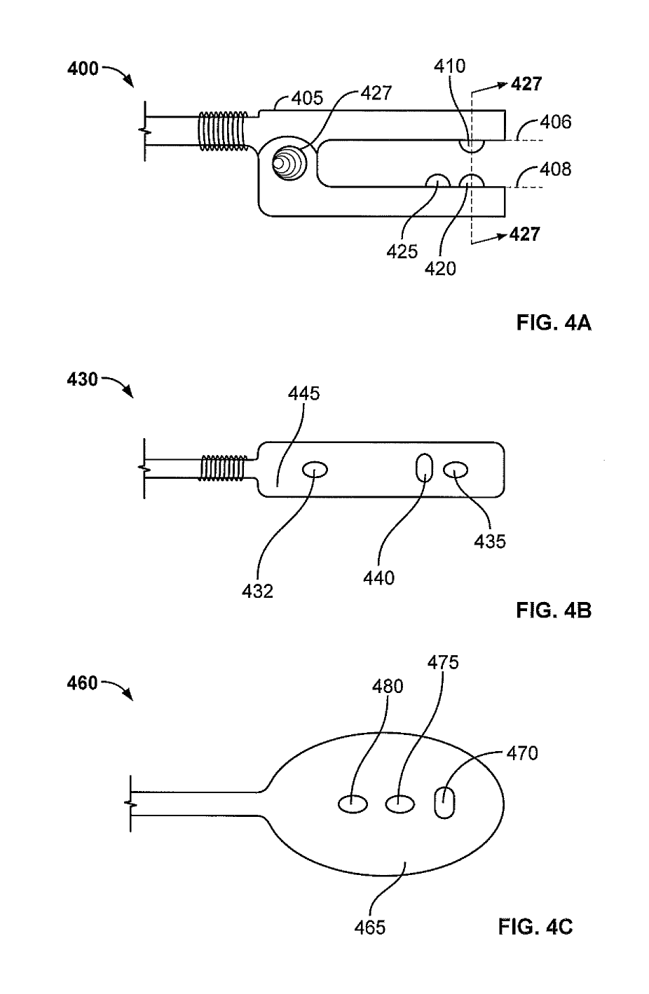

FIGS. 4A-4C depict illustrative arrangements of combined PPG-EKG sensors that may be used in a monitoring system such as monitoring system 100 (FIG. 1), 200 (FIG. 2), and/or 300 (FIG. 3). Sensors 400, 430, and 460 each illustrate particular sensor designs that combine PPG and EKG capable detectors. Each of sensors 400, 430, and 460 may be similar or identical to, for example, sensor 110 (FIG. 1), sensor 120 (FIG. 1), sensor 210 (FIG. 2), sensor 220 (FIG. 2), sensor 312 (FIG. 3), and/or sensor 346 (FIG. 3). Further, although sensors 400, 430, and 460 are sometimes described as being variously digit-, or forehead-based sensors, and in some cases, disposable, it will be understood that these sensors may each be modified to be affixed on other parts of a subject's body (such as a subject's ear, or any other suitable body part) and/or may be adapted to be reusable (long-term use) sensors. For example, any of sensor 400, 430, and 460 may be used on the ear of a subject. The PPG detection capability of sensors 400, 430, and 460 may be used to, for example, determine the blood oxygen saturation and/or blood pressure of a subject. The blood pressure of a subject may be determined, for example, by applying CNIBP-based techniques to an obtained PPG signal or signals.

FIG. 4A illustrates a reusable sensor designed to be affixed onto a subject's digit, appendage, or any other suitable body part, using clip mechanism 405. Sensor 400 may include a spring or other flexible device to adjust the width of the sensor opening. For example, sensor 400 may include spring 427, which may be an adjustable torsion spring or any other suitable type of spring. In an arrangement, spring 427 may be used to bias the size of the opening between upper contact surface 406 and lower contact surface 408. Further, spring 427 may allow the opening between upper contact surface 406 and lower contact surface 408 to rotate or displace up and/or down in order to accommodate the size and shape of a subject's digit, appendage, or other suitable body part or parts. Sensor 400 may include a padded surface, for example, made of foam or a foam-like material, along upper contact surface 406 and/or lower contact surface 408 to provide a comfortable fit to a subject such as subject 105 (FIG. 1). In an arrangement, sensor 400 may detect a PPG signal using LED 410 and photodetector 420. In an arrangement, sensor 400 may include an EKG contact sensor (e.g., any suitable electrode) such as EKG electrode 425 to detect electrical activity of a subject's heart. EKG electrode 425 may be placed anywhere in, on, or around sensor 400, and the location of EKG electrode 425 may be chosen to provide a strong measurement of the electrical signals related to the activity of the subject's heart. In an advantageous arrangement, EKG electrode 425 may be placed on lower contact surface 408. In this configuration, EKG electrode 425 may be less likely to placed on top of a subject's nail (the fingernail and toenail typically not providing a strong EKG signal) and is more likely to come into contact with a moist portion of the subject's digit (a moist contact surface typically providing strong EKG measurements).

In an arrangement, any number of sensors similar or identical to sensor 400 may be placed on a subject, such as subject 105 (FIG. 1) to perform joint PPG and EKG measurements. In an arrangement, some sensors may contain only EKG functionality and other sensors may contain only PPG functionality. For example, a total of twelve sensors similar or identical to sensor 400 may be affixed to a subject on a digit, appendage (such as an ear), any other suitable body part, or any suitable combination thereof. In an arrangement, only two of these twelve sensors may be used to determine a PPG signal (and from these PPG signals, determine a subject blood pressure), while all twelve sensors may be used to determine EKG, and, for example, the presence of a subject arrhythmia.

FIG. 4B illustrates a disposable (e.g., one-time use) sensor designed to be affixed onto a subject's digit, appendage, or any other suitable body part using an adhesive patch-like surface. Sensor 430 may be shaped similar to, for example, a rectangular bandage. In an arrangement, adhesive surface 445 may be covered by a laminate or other protective surface, and an operator or a subject may remove this protective surface prior to use to expose adhesive surface 445. Sensor 430 may then be “wrapped” around a digit of the subject. In an arrangement, sensor 430 may detect a PPG signal using LED 432 and photodetector 435. In an arrangement, the spacing between LED 432 and photodetector 435 may be designed based at least in part on an average or typical and/or representative human digit so that it is likely that LED 432 and photodetector 435 will be directly opposed to each other once sensor 430 is wrapped around a subject's digit. In an arrangement, a variety of sensors sizes may be manufactured, and, for example, a medical professional may choose the appropriate sized version of a sensor similar or identical to sensor 430 to provide to a subject. In an arrangement, sensor 430 may include instructions to first place photodetector 435 on the bottom of a subject's digit and then wrap sensor 430 around the subject's digit thereby ensuring that photodetector 435 is advantageously placed on the bottom of a subject's digit. Sensor 430 may include an EKG contact sensor (e.g., any suitable electrode) such as EKG contact sensor 440 to detect electrical activity of a subject's heart. EKG contact sensor 440 may be advantageously placed near photodetector 435, as depicted in FIG. 4B, so as to increase the likelihood that EKG contact sensor 440 is placed on a portion of the subject's digit that does not include the subject's nail.

In an arrangement, any number of sensors similar or identical to sensor 430 and/or sensor 400 (FIG. 4A) may be placed on a subject, such as subject 105 (FIG. 1) to perform joint PPG and EKG signal measurements. In an arrangement, some of these sensors may contain only EKG functionality and other sensors may contain only PPG functionality. For example, a total of twelve sensors similar or identical to sensor 430 and/or sensor 400 (FIG. 4A) may be affixed to a subject. In an arrangement, only two of these twelve sensors may be used to measure a PPG signal (and from the PPG signal, determine a subject blood pressure), while all twelve sensors may be used to determine EKG, and, for example, the presence of a subject arrhythmia. Alternatively or additionally, one of the twelve sensors may be used to determine a subject oxygen saturation and/or blood pressure (while all twelve sensors may be used to determine EKG).

FIG. 4C illustrates a disposable (e.g., one-time use) sensor designed to be affixed onto a exposed area of a subject's body, such as a subject's forehead (and may also be attached to a subject's digit, or any other suitable body part). Sensor 460 includes an adhesive patch-like surface, and may be shaped similar to, for example, a bandage having an approximate or exact oval shape. In an arrangement, adhesive surface 465 may be covered by a laminate or other protective surface, and an operator or a subject may remove this protective surface to expose adhesive surface 465 prior to use. Sensor 465 may then be placed on the forehead of the subject. In an arrangement, sensor 465 may detect a PPG signal using LED 480 and photodetector 475. In an arrangement, a variety of sensor sizes may be manufactured, and, for example, a medical professional may choose the appropriate sized version of a sensor similar or identical to sensor 460 to provide to a subject.

Sensor 460 may include an EKG contact sensor (e.g., any suitable electrode) such as EKG contact sensor 470 to detect electrical activity of a subject's heart. In an arrangement, any number of sensors similar or identical to sensor 460, sensor 430 (FIG. 4B), and/or sensor 400 (FIG. 4A) may be placed on a subject, such as subject 105 (FIG. 1) to perform joint PPG and EKG measurements. In an arrangement, some of these sensors may contain only EKG functionality and other sensors may contain only PPG functionality. For example, a total of twelve sensors similar or identical to sensor 460, sensor 430 (FIG. 4B), and/or sensor 400 (FIG. 4A) may be affixed to a subject. In an arrangement, only two of these twelve sensors may be used to determine a PPG signal (and from the PPG signal, determine a subject blood pressure and/or oxygen saturation level), while all twelve sensors may be used to determine EKG, and, for example, the presence of a subject arrhythmia.

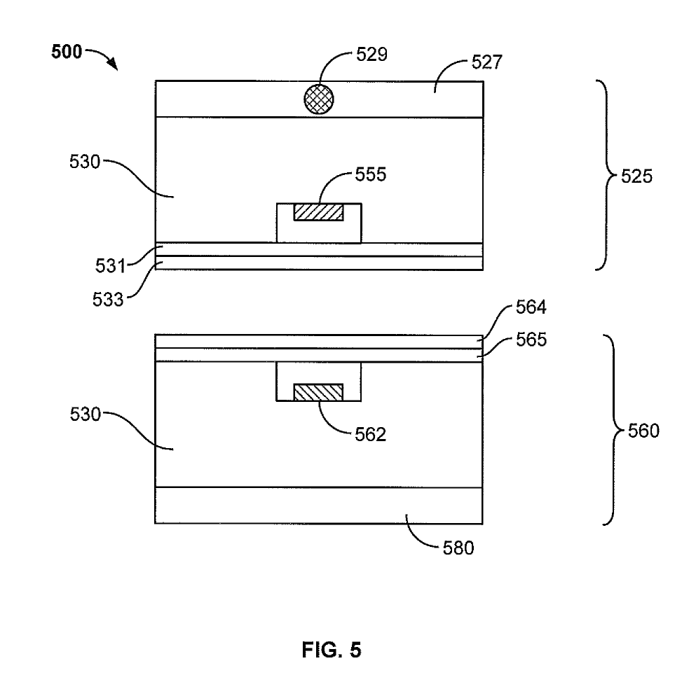

FIG. 5 depicts an illustrative cross-sectional view of a combined PPG-EKG sensor unit that may be used in a monitoring system such the monitoring system depicted in any of FIGS. 1-3. Sensor unit 500 may depict a cross-sectional view of any of the sensors of, for example, FIGS. 4A-C. In an arrangement, sensor unit 500 may depict a cross-sectional view of sensor 400 (FIG. 4A), taken along plane 427 (FIG. 4A).

Sensor unit 500 may be designed to, for example, attach to a digit, appendage (e.g., an ear), or other part of a subject. In an arrangement, sensor unit 500 may include top part 525 and bottom part 560. Top part 525 may correspond to, for example, the top half of sensor 400 (FIG. 4A), and bottom part 560 may correspond to the bottom half of sensor 400 (FIG. 4A). In an arrangement, top part 525 may be configured to attach or conform to the top (or “top side”) of a subject's digit, and bottom part 560 may be configured to attach to the bottom (or “bottom side”) of the subject's digit. For example, top part 525 and/or bottom part 560 may include padding material, where the padding material is capable of conforming to the subject's digit. In an arrangement, the padding material may be made of a foam or foam-like material, that adjusts to the shape and/or size or a subject's digit.

In an arrangement top part 525 and bottom part 560 may be connected and/or bound together by a support structure such as support structure 530. Support structure 530 may include one or more cables connected to the PPG and EKG sensors and may receive and/or hold these cables in place. Support structure 530 may provide general support for several constituent components of sensor unit 500 (to be described in the following). Support structure 530 may be made from, for example, foam or any other suitable flexible, amorphous, and/or malleable material. In an arrangement, support structure 530 may adapt, connect, and/or otherwise conform to the digit, appendage (e.g., an ear), or other part of a subject. In an arrangement, the material of support structure 530 may be more flexible (and/or more malleable and/or more amorphous) closer to the intended contact point with the digit, appendage (e.g., an ear), or other part of the subject, and may then become progressively less flexible (and/or less malleable and/or less amorphous) at points further away from this intended contact point.

Sensor unit 500 may include adhesive layer 531 and/or 565. Adhesive layer 531 (and similarly, adhesive layer 565) may consist of a sticky, glue-like material that naturally bonds with the contact surface of the subject. For example, in an arrangement, adhesive layer 531 (and similarly, adhesive layer 565) may be similar or identical to the adhesive material typically found in common medical bandages. In an arrangement, the material of adhesive layer 531 (and similarly, adhesive layer 565) may be formulated and/or chosen specifically to form a solid contact with surfaces having the properties of human skin (for example, based at least in part on an expected amount of moisture in human skin). Sensor unit 500 may include non-stick layer 533 and/or 564. Non-stick layers 533 and 564 may be formulated from a substance or material designed to protect underlying adhesive layers, for example, adhesive layers 531 and 565, respectively. For example, in an arrangement, the material of adhesive layer 531 and/or 565 may resemble a non-stick “peel” similar or identical to that found in many common medical bandages. Adhesive layer 531 and/or 565 may be designed to be removed (e.g., “peeled off”) from sensor unit 500 prior to attachment of sensor unit 500 to a subject.

Sensor unit 500 may include cover material 527 and cover material 580. Cover material 527 and cover material 580 may each be made of a hard plastic or other rigid material and/or compound, and may be designed or chosen to protect the various components of sensor unit 500 described above. Sensor unit 500 may include interface 529. Interface 529 may be coupled to one or more sensors, for example, to sensor 555 and/or sensor 562, and may be used transmit or otherwise provide signals detected by these sensor to one or more electronic monitors. Interface 529 may correspond to, for example, a cable such as a data cable and/or to a wireless transmitter.

Sensor unit 500 may include sensor 555 and/or sensor 562. Each of these sensors may correspond to, for example, a partial or full PPG or EKG sensor. For example, in an arrangement, sensor 555 (and similarly, sensor 562) may correspond to a part of a PPG sensor, such as LED 410 (FIG. 4A), 432 (FIG. 4B), or 480 (FIG. 4C), or photodetector 420 (FIG. 4A), 435 (FIG. 4B), or 475 (FIG. 4C). Alternatively, sensor 555 may correspond to a part of an EKG sensor, such as EKG electrode 425 (FIG. 4A), 440 (FIG. 4B), or 470 (FIG. 4C). In an arrangement, sensor 555 may correspond to an EKG electrode that includes an electrically conductive adhesive or adhesives, such as an Aquagel or Hydrogel. Additionally, or alternatively, an operator (for example, a end-user or a medical professional) may apply such a conductive adhesive directly to a part of a subject (such as a suitable digit, appendage, or other body part of the subject) on which the sensor is to be attached. Sensor unit 500 may include additional sensors and/or parts of sensors, such as PPG or EKG sensors, that are not depicted in the cross-sectional view of FIG. 5.

It will be understood that sensor unit 500 is merely an illustrative sensor unit, and that portions of sensor unit 500 may be omitted without completely excluding the disclosed concepts and techniques. For example, in an arrangement, sensor unit 500 may omit adhesive layer 531 and/or 565. In such an alternative arrangement, sensor unit 500 may be connected or fastened to a subject, for example, using a clip and/or spring mechanism. The clip and/or spring mechanism may be fastened by tension, gravity, by any other suitable technique, or by any suitable combination thereof. Similarly, non-stick layer 533 and/or 564 may be omitted in an arrangement of sensor unit 500. In an arrangement, more than one interface, including, for example, a combination of any suitable number of cables and/or wireless links, may be used to connect sensor unit 500 to, for example, more than one electronic monitor.

It will also be understood that the above method may be implemented using any human-readable or machine-readable instructions on any suitable system or apparatus, such as those described herein.

The foregoing is merely illustrative of the principles of this disclosure and various modifications can be made by those skilled in the art without departing from the scope and spirit of the disclosure. The following claims may also describe various aspects of this disclosure.

What is claimed is:

1. A sensor unit for detecting signals related to physiological characteristics of a subject, the sensor unit comprising:
a support structure;
a first sensor capable of detecting a photoplethysmograph (PPG) signal, wherein the first sensor is physically coupled to the support structure;
a second sensor capable of detecting an electrocardiographic (EKG) signal, wherein the second sensor is physically coupled to the support structure; and
an interface coupled to the first sensor and the second sensor, wherein the interface is capable of providing the detected PPG signal and the detected EKG signal to at least one electronic monitor.

2. The sensor unit of claim 1, wherein the first sensor comprises a LED emitter and a photoelectric detector.

3. The sensor unit of claim 1, wherein the second sensor comprises a metal electrode.

4. The sensor unit of claim 1, wherein the sensor unit is configured to be attached to a subject's digit, and wherein the sensor unit further comprises:
a first part configured to contact the top of the digit; and
a second part configured to contact the bottom of the digit, wherein the second sensor is coupled to a portion of the support structure that is located in the second part of the sensor and is arranged to contact the underside of a digit of the subject.

5. The sensor unit of claim 1, wherein the sensor unit is configured to be attached to a subject's digit, and wherein the sensor unit further comprises:
a first part configured to contact the top of the digit; and
a second part configured to contact the bottom of the digit, wherein the second sensor is coupled to a portion of the support structure that is located in the first part of the sensor and is arranged to contact the top of the digit at a location separate from a nail of the subject.

6. The sensor unit of claim 1, wherein the sensor further comprises an adhesive layer, wherein the adhesive layer is physically coupled to the support structure, and wherein the adhesive layer is arranged to be affixed onto a portion of the body of the subject.

7. The sensor unit of claim 1, further comprising:
a clip comprising a spring, wherein the spring is capable of biasing the support structure; and
wherein the support structure comprises padding material, wherein the padding material is adjustable to conform to a digit of the subject.

8. The sensor unit of claim 1, wherein the interface is a wireless interface capable of providing the detected PPG signal and the detected EKG signal to the at least one electronic monitor by a wireless link.

9. The sensor unit of claim 1, further comprising:
a first cable coupled to the interface for providing the detected PPG signal to the at least one electronic monitor; and
a second cable coupled to the interface for providing the detected EKG signal to the at least one electronic monitor.

10. The sensor unit of claim 1, further comprising a common cable capable of providing both the detected PPG signal and the detected EKG signal to the at least one electronic monitor.

11. A monitoring system for determining one or more physiological parameters of a subject, the system comprising:
a sensor unit, the sensor unit comprising:
a support structure;
a first sensor capable of detecting a photoplethysmograph (PPG) signal, wherein the first sensor is physically coupled to the support structure;
a second sensor capable of detecting an electrocardiographic (EKG) signal, wherein the second sensor is physically coupled to the support structure; and
an interface coupled to the first sensor and the second sensor, wherein the interface is capable of providing the detected PPG signal and the detected EKG signal to at least one electronic monitor; and
a processor coupled to the at least one electronic monitor, wherein the processor is capable of:
determining the one or more physiological parameters of the subject based, at least in part, on the detected PPG signal;
determining an auxiliary parameter based, at least in part, on the detected EKG signal; and
outputting the one or more physiological parameters.

12. The system of claim 11, wherein the determining of the one or more physiological parameters of the subject is performed using the auxiliary parameter.

13. The system of claim 11, wherein the auxiliary parameter comprises a trigger, and wherein the processor is further capable of generating an ensemble average of the determined PPG signal in response to the trigger.

14. The system of claim 11, wherein the auxiliary parameter comprises an indicator of the presence of an arrhythmia condition in the subject.

15. The system of claim 11, wherein the auxiliary parameter comprises respiration information determined based at least in part on at least one impedance change of the subject.

16. A method for determining one or more physiological parameters of a subject, the method comprising:
detecting a photoplethysmograph (PPG) signal;
detecting an electrocardiographic (EKG) signal;
providing the detected PPG signal and the detected EKG signal to at least one electronic monitor;
determining the one or more physiological parameters of the subject based, at least in part, on the detected PPG signal;
determining an auxiliary parameter based, at least in part, on the detected EKG signal; and
outputting the one or more physiological parameters.

17. The method of claim 16, wherein the determining of the one or more physiological parameters of the subject is performed using the auxiliary parameter.

18. The method of claim 16, wherein the auxiliary parameter comprises a trigger, the method further comprising generating an ensemble average of the detected PPG signal based, at least in part, on the trigger.

19. The method of claim 16, wherein the auxiliary parameter comprises an indicator of the presence of an arrhythmia condition in the subject.

20. The method of claim 16, wherein the auxiliary parameter comprises respiration information determined based at least in part on at least one impedance change of the subject.
